(19) United States
(12) Patent Application Publication
(10) Pub. No.: US 2024/0041630 A1
(43) Pub. Date: Feb. 8, 2024

(54) WEARABLE APPARATUS FOR EXERCISE AND POSTURE CORRECTION

(71) Applicant: Venesa Sullivan, Columbus, OH (US)

(72) Inventor: Venesa Sullivan, Columbus, OH (US)

(21) Appl. No.: 18/230,235

(22) Filed: Aug. 4, 2023

Related U.S. Application Data
(60) Provisional application No. 63/395,383, filed on Aug. 5, 2022.

(51) Int. Cl. A61F 5/02; A63B 21/04; A63B 21/00; A63B 23/035; A63B 23/04; A63B 23/12

(52) U.S. Cl. CPC A61F 5/026; A63B 21/0442; A63B 21/4007; A63B 21/4033; A63B 23/03541; A63B 23/04; A63B 23/12; A63B 2225/09

(57) ABSTRACT

An apparatus is described that involves a harness with attached, adjustable resistance elements. In one example of the apparatus of the present invention, the resistant elements provide resistance to various user body parts, such as, for example, user limbs, glutes, chest, back and/or core, and accordingly, passive exercise accompanies normal movement of the user. The apparatus may be useful for various functions when a user is going about the routine tasks and activities of daily life, and/or may be useful for creating or supplementing user exercise plans. The apparatus may also be useful for promoting posture correction.

### CROSS-REFERENCE TO RELATED APPLICATIONS

This non-provisional application claims priority to U.S. Provisional Application No. 63/395,383, filed Aug. 5, 2022, the contents of which are hereby incorporated by reference as if fully recited herein.

### TECHNICAL FIELD

The disclosed invention relates to an exercise apparatus, and more particularly to a wearable apparatus with resistance elements that provide resistance to the appendages of a wearer, so that passive exercise accompanies normal movement of the wearer, including posture correction. The wearer may selectively use the apparatus while going about the routine tasks and activities of daily life, and/or may customize or supplement an active exercise program through the resistance elements of the apparatus. The wearer may also selectively use the apparatus excluding resistance elements to specifically provide posture correction.

### BACKGROUND AND SUMMARY OF THE INVENTION

Muscles operate by being selectively extended or contracted. Because a muscle has its respective ends attached to different skeletal features, the extension or contraction causes relative motion of the skeletal feature. Muscle tone is built and maintained by repeated action of the various muscles and muscle groups.

In an active exercise program, the exercising person uses resistance, gravity (such as by carrying or moving weights), or a combination of resistance and gravity, to oppose the muscle being exercised. In many of these instances, a device external to the body of the exercising person provides the opposition to the muscle or muscle group. This can be an exercise machine or a static feature, such as a wall or a barre.

In some instances, the exercising person actively uses portions of their own body to provide the opposition or resistance. An example is the use of a resistance band to exercise a leg and/or an arm of the exercising person.

Active exercise may be very useful, provided that the exercising person has the time and opportunity to engage in it. However, the prior art does not provide effective ways for a person going about the routine tasks and activities of daily life (e.g., walking, running, chores, shopping, sports, training, entertainment, physical therapy activities, some combination thereof, or the like) from enhancing those movements by, for example, effectively converting their trunk and/or torso into a resistance device that provides opposition to the person's muscle(s) or muscle group(s). It is a goal of the present invention to allow for such passive enhancement.

The aforementioned goal and other objectives are achieved by an exemplary wearable apparatus for exercise and posture correction.

According to the present invention in one aspect, an exemplary apparatus, wearable by a user, comprises a harness device. The harness device may be configured to be worn on an upper torso. The harness device may have a plurality of straps that may be adjustable to provide targeted pressure. The targeted pressure may achieve posture correction of the user by aligning the user's back and shoulders.

The apparatus may further include a plurality of resistance elements, each of which may comprise a length of elastomeric material with a fastening means affixed at each end thereof.

The apparatus may also include a plurality of straps, configured for receiving a hand or foot of the user, each strap having a fastening means complementary to the fastening means of the resistance elements. The apparatus may additionally include a plurality of fastening means, complementary to the fastening means on the resistance elements, arranged on the harness device.

The user may exercise one or more appendages by anchoring one end of one of the resistance elements to the harness device, and inserting a hand or foot into a strap attached at an opposite end of the resistance element.

### BRIEF DESCRIPTION OF THE DRAWINGS

Novel features and advantages of the present invention, in addition to those expressly mentioned herein, will become apparent to those skilled in the art from a reading of the following detailed description in conjunction with the accompanying drawings. The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 is a front perspective view of an exemplary embodiment of a wearable apparatus for exercise and posture correction;

FIG. 2 is a rear perspective view of the FIG. 1 embodiment;

FIG. 3 is an isolated front view of an exemplary harness device of the FIG. 1 embodiment;

FIG. 4 is an isolated view of an exemplary arm resistance element for exercising an arm of a user;

FIG. 5 is a perspective view of the FIG. 4 arm resistance element as worn by the user;

FIG. 6 is an isolated view of an exemplary leg resistance element for exercising a leg of the user;

FIG. 7 is a perspective view of the FIG. 6 leg resistance element as worn by the user; and

FIG. 8 is an isolated view of an optional, supplemental interchangeable arm/leg resistance element.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

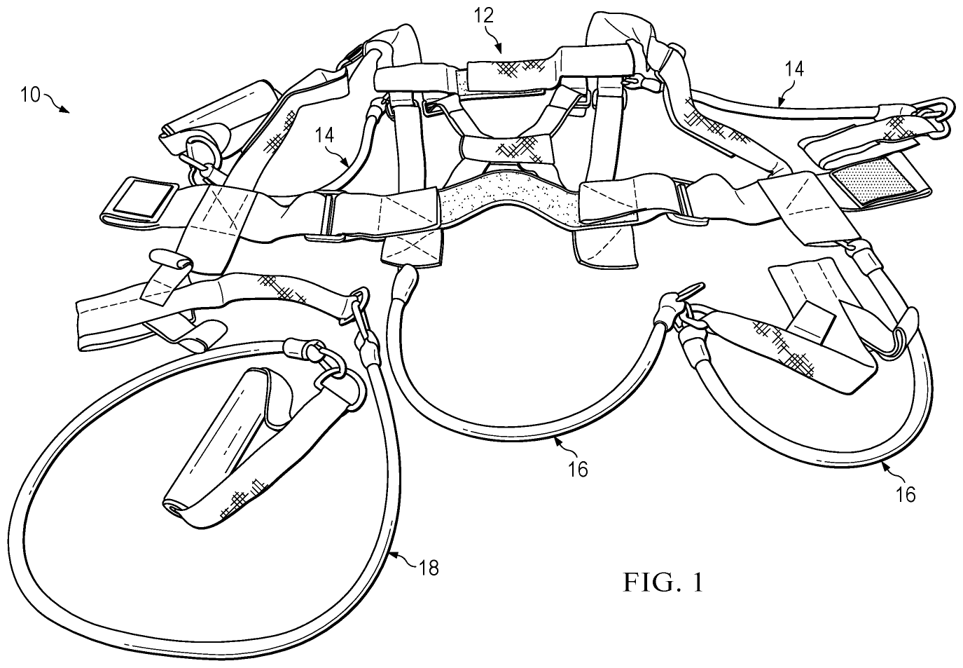

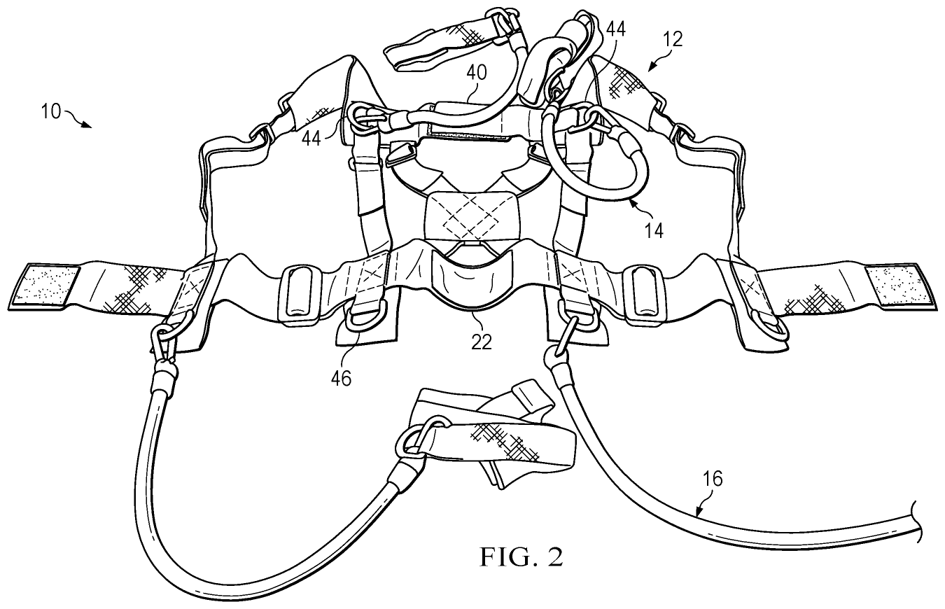

FIGS. 1 and 2 show an embodiment 10 of a wearable exercise apparatus, with FIG. 1 being a front perspective view and FIG. 2 being a rear perspective view thereof. The embodiment 10 as depicted has a harness device 12, a pair of arm resistance elements 14; a pair of leg resistance elements 16; and a supplemental resistance element 18, which may be considered an optional feature of the apparatus 10. While the supplemental resistance element is seen in FIG. 1, it is not shown in FIG. 2. It will be apparent to one of ordinary skill in the art that the particular number, material composition, location, and the like of exemplary resistance elements may be varied without departing from the scope of the present invention.

Each resistant element (e.g., 14, 16, 18) may be secured to the harness 12 at a connection point affixed to the harness 12. The connection point may comprise a closed loop (e.g., ring, carabiner, or the like), clip, buckle, some combination thereof, or the like. It will be apparent to one of ordinary skill in the art that variations to the type and particular location of a connection point may be made without departing from the scope of the present invention. In the FIG. 2 embodiment, scapula closed loops 44 are positioned on each side of the harness 12, and are each configured to permit an arm resistance element 14 to be attached to the harness.

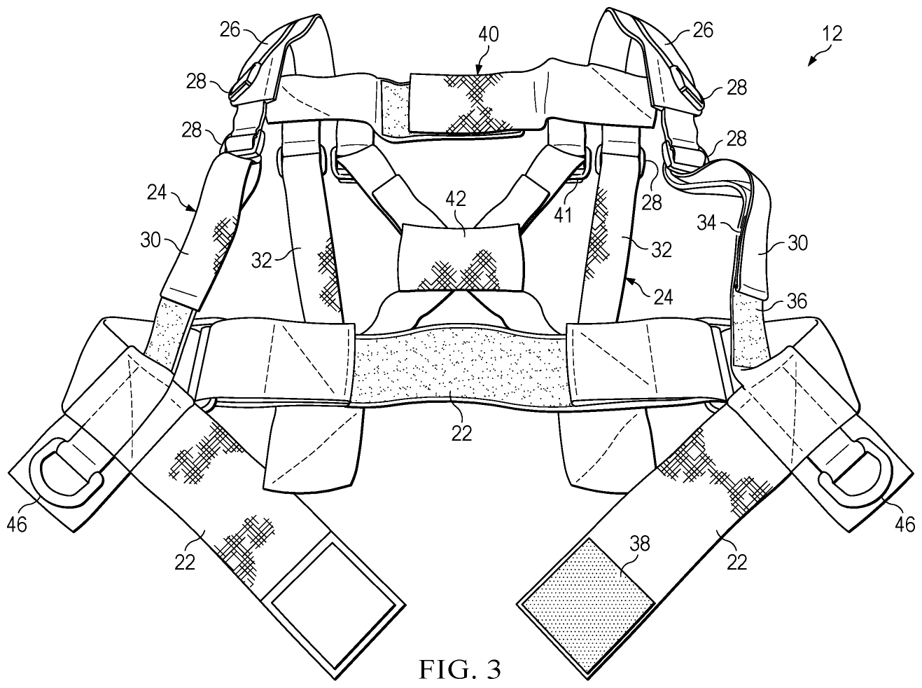

Harness device 12 is shown in FIG. 3 in an isolated front view. As is recognizable from its features, harness device 12 may be worn on an upper torso of the user. Harness device 12 may have five distinct connecting adjustable sections. The harness device 12 may use the torso of a user as an anchor for the various resistance elements. As the harness device 12 may be used by people of different size, age, sex and body type, etc., it is a critical feature of the harness device 12 of this particular embodiment that it be readily adjustable. To that end, the harness device 12 may comprise a horizontal strap 22, having three parts, to be fitted around a lower chest area of the user, and two pairs of vertical straps 24.

In this particular embodiment, each of a first pair of the vertical straps 24 is intended to pass from a portion of the horizontal strap 22 upward along the back of the user, through a belt loop 28 on a back end of a shoulder strap 26. Additionally, here, each of a second pair of vertical straps 24 is intended to pass from a portion of the horizontal strap 22 upward along the chest of the user and through one of the two belt loops 28 on a front end of a shoulder strap 26. In a preferred embodiment of the harness device 12, an end of each of a first pair of vertical straps 24 is attached in a fixed manner, such as, for example, by sewing, to the horizontal strap 22 along the back of the user. An end of each of a second pair of vertical straps 24 is attached in a fixed manner, such as, for example, by sewing, to the horizontal strap 22 along the front of the user. However, to provide the desired adjustability, each vertical strap 24 may be provided with at least one means for adjustment, preferably along each of the portions along the front and the back of the user.

In the depicted embodiment, the vertical straps 24 present both a front and a back adjustment means. Each vertical strap 24 may comprise three parts. A first of these parts may be a shoulder strap 26 having, for example, at least two belt loops 28, one on an end of the front side and one on an end of the back side. The shoulder strap 26 may comprise another belt loop 28 independent from the aforementioned ends for additional strap adjustment, but such is not required. The second and third parts of each vertical strap 24 may comprise a front adjustable element 30 and a rear adjustable element 32. In FIG. 3, each of the front and rear adjustable elements 30, 32 are depicted as being fixedly attached at one end thereof to a corresponding portion of the horizontal strap 22. A second end of each of the front and rear adjustable elements 30, and 32 may be arranged to pass up and through a belt loop 28, and to be adjustably fixed to itself. In FIG. 3, this is shown by placing a first part 34 of a hook-and-loop fastener at the second end of the adjustable element, and a corresponding second part 36 of the hook-and-loop fastener. Other corresponding fastener means may be used, such as, for example, a belt buckle at the second end of the adjustment element that attaches through a series of holes provided along the length of the adjustment element.

In a similar manner, the horizontal strap 22 may comprise any number of different types of connecting adjustable elements. It is most commonly expected to be configured as a belt having corresponding hook-and-loop fastener parts, such as, for example, the loop portion 38 shown in FIG. 3. The corresponding hook portion is not visible in FIG. 3, as it is on the rear surface of the opposing end of the horizontal strap 22.

There are further elements of the harness device 12 that are shown in FIG. 3. A first of these is an exemplary strap 40 comprising two sections arranged to pass across, and connect in the middle of, the upper scapula region of the user's back, joining the shoulder straps 26 to each other, preferably in an adjustable manner. Each section may be attached to the shoulder straps 26 in a fixed manner, such as, for example, by sewing. Strap 40 may run substantially parallel to the horizontal strap 22. The second of said further elements of the FIG. 3 embodiment is an “X”-shaped strap arrangement 42 that crisscrosses the thoracic portion of the user's back. Each lower end of the strap arrangement 42 may be attached to the horizontal strap 22 along the lower back of the user, in a fixed manner, such as, for example, by sewing. Each upper end of the strap arrangement 42 may pass up the upper back and through a belt loop 41 proximate to each outward end of the strap 40. Preferably, the upper ends of the strap arrangement 42 are provided with fastening means, especially hook and loop fastening means that can adjustably connect the strap arrangement 42 to the strap 40. Referring now to FIGS. 1-3, the structure established and maintained by the harness device 12 may allow the harness device to provide significant posture correction to a user, even when it is used without the resistance elements 14, 16.

As mentioned above, the harness device 12 has a primary intention to anchor resistance elements. Here, the depicted embodiment achieves this by placing a series of closed loops (44, 46) for removable attachment of a clip on the resistance elements. In FIG. 2, a first pair of such closed loops 44 are shown on the outer ends of the scapula strap 40 and a second pair of the closed loops 46 are shown along the horizontal strap 22, in the lower rear thoracic region of the user. FIG. 3 shows a third pair of such closed loops 46 on the horizontal strap 22, in the lower front thoracic region of the user.

The various straps of the harness device 12 will typically be formed from lengths of a woven web of fabric. A typical width, depending upon the specific strap, will be in the range from 1 to 2 inches. The web material may be polymeric, such as, for example, a polyamide or polypropylene, but may additionally or alternatively be a natural renewable material, such as, for example, cotton. At least portions of some of the straps may include elastic material. It is preferred that the fabric used be machine washable.

Referring now to FIGS. 1-4, as depicted, the harness device 12 is most likely worn by a user on top of a conventional garment, but it may be worn underneath a garment, preferably as long as the resistance elements are attached to the closed loops 44, 46, 54 prior to placing the garment over the harness device. However, when the harness device 12 is worn as a posture correction device, the use of resistance elements is not required.

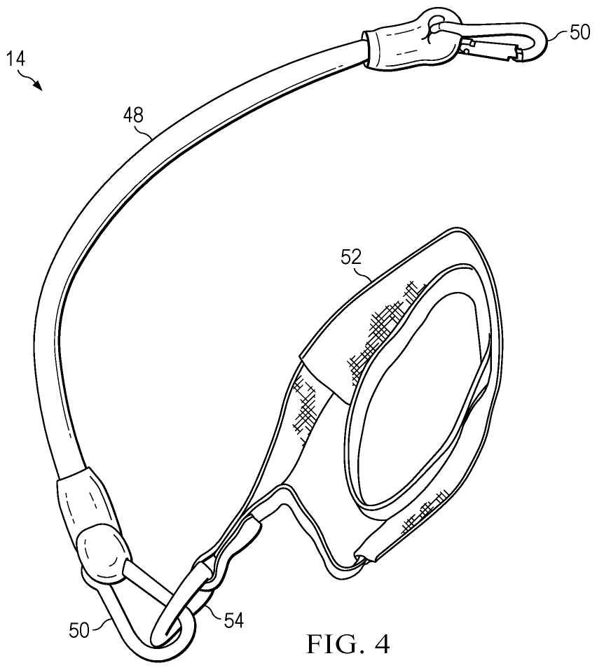

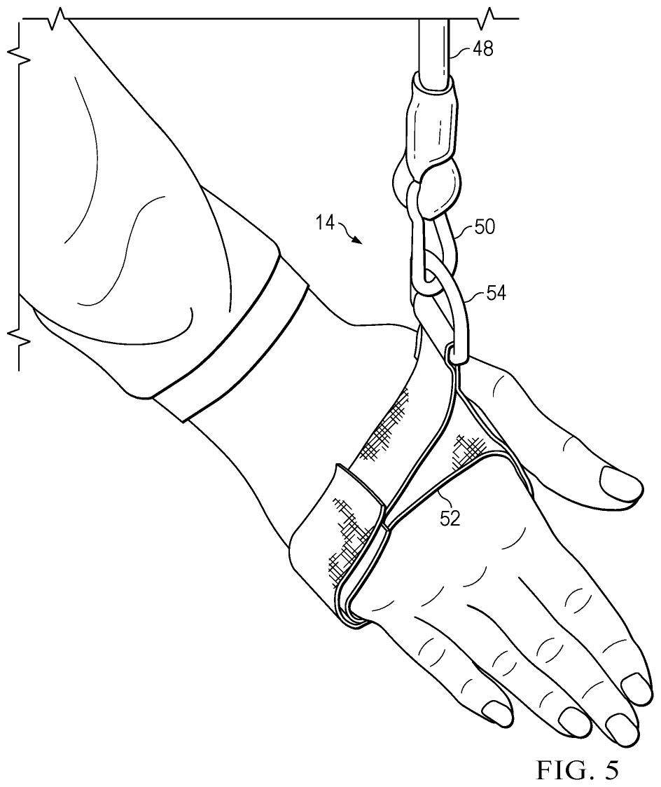

FIGS. 4 and 5 depict a first type of resistance element, specifically the arm resistance element 14, in an isolated view (FIG. 4) and in use by a user (FIG. 5). The most notable feature of the resistance element 14 is a length of an elastomeric material 48, provided at each end with a fastener loop or clip 50. Preferably, both ends are provided with a clip 50 that is, e.g., suitable for attachment to one of the closed loops 44 shown and described with regard to FIG. 2. The length and diameter of the elastomeric material may be varied and selected by the individual user, as the amount of resistance against a length-wise stretching force may be varied. While it would be possible to use a loop of resistance band, as is known in exercise equipment, the preference would seem to be to use a hollow length of elastomeric tubing. Such tubing is commonly available as an exercise device, with different diameters often being used to provide the different levels of resistance. In many cases, the elastomeric tubing, which may be a natural rubber product, synthetic material, some combination thereof, or the like, is color-coded to designate the resistance level.

In addition to the length of elastomeric material 48 and the end clips 50, each arm resistance element 14 may include a hand strap 52 for adjustable passage around the palm of the user, preferably, as shown, with the fingers and thumb not constrained by the strap 52. A closed loop 54 attached to the hand strap 52 may permit the hand strap to be removably attached to one of the end clips 50 of elastomeric material 48.

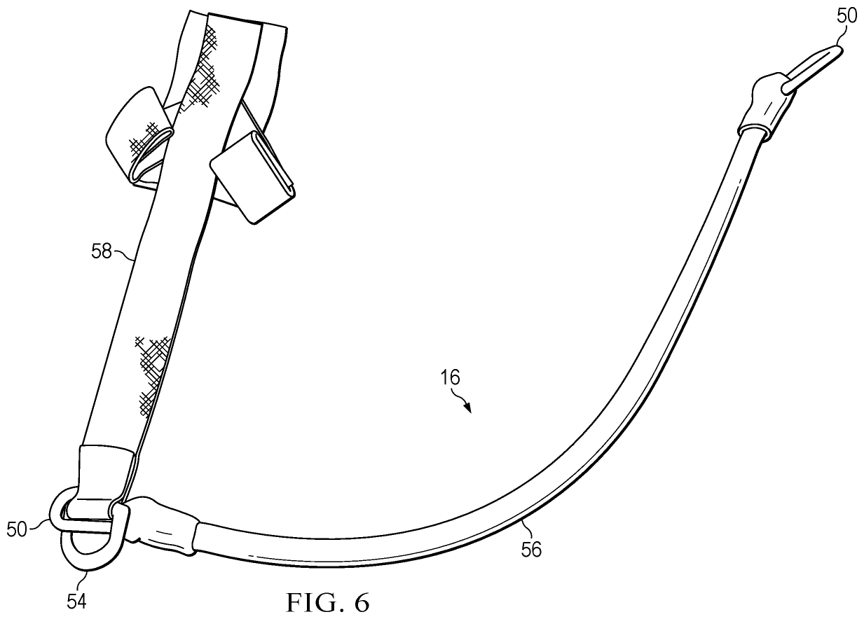

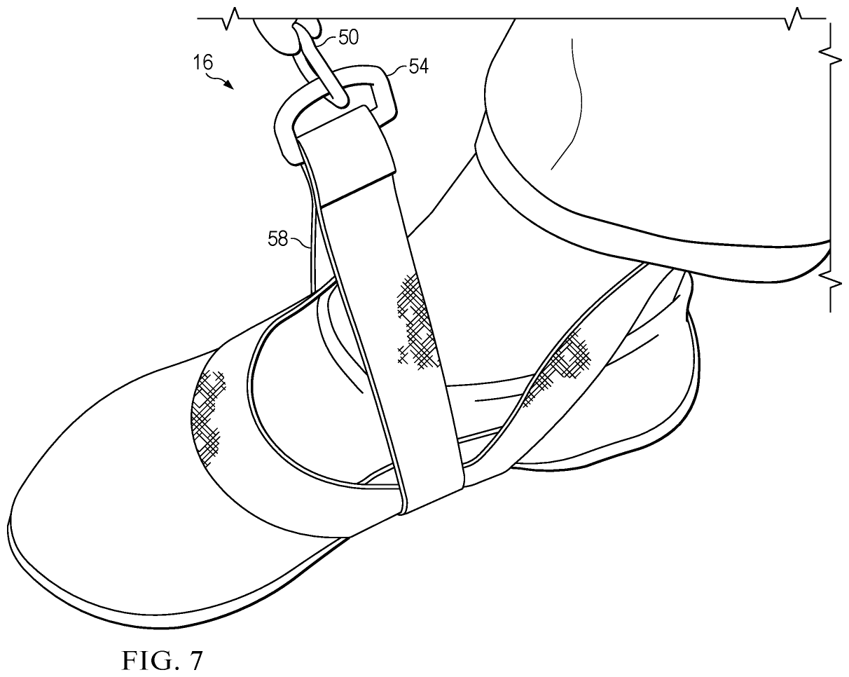

In an analogous manner to the arm resistance element of FIGS. 4 and 5, FIGS. 6 and 7 depict a second type of resistance element, specifically the leg resistance element 16, in an isolated view (FIG. 6) and in use by a user (FIG. 7). The most notable feature of the leg resistance element 16 is a length of an elastomeric material 56, provided at each end with a fastener loop or clip 50. Preferably, both ends are provided with a clip that is, e.g., suitable for attachment to one of the lower chest closed loops 46 shown and described with regard to FIGS. 2 and 3. The length and diameter of the elastomeric material may be varied and selected by the individual user, to, e.g., provide a selected amount of resistance against a length-wise stretching force. While it would be possible to use a loop of resistance band, as is known in exercise equipment, the preference would seem to be to use a hollow length of elastomeric tubing. Such tubing is commonly available as an exercise device, with different diameters often being used to provide the different levels of resistance. In many cases, the elastomeric tubing, which may be a natural rubber product, a synthetic material, some combination thereof, or the like, is color-coded to designate a resistance level. In contrast to the elastomeric tubing used in the arm resistance element, one would generally use a longer length of the tubing for the leg resistance element, and would generally select a higher level of resistance for the legs, due to the mass of the muscles being exercised.

In addition to the length of elastomeric material 56 and the end clips 50, each leg resistance element 16 may include a foot strap 58 for, e.g., adjustable passage around the mid-sole of the user's foot, either with or without footwear. A closed loop 54 attached to the foot strap 58 may permit the foot strap to be removably attached to one of the end clips 50 of elastomeric material 56.

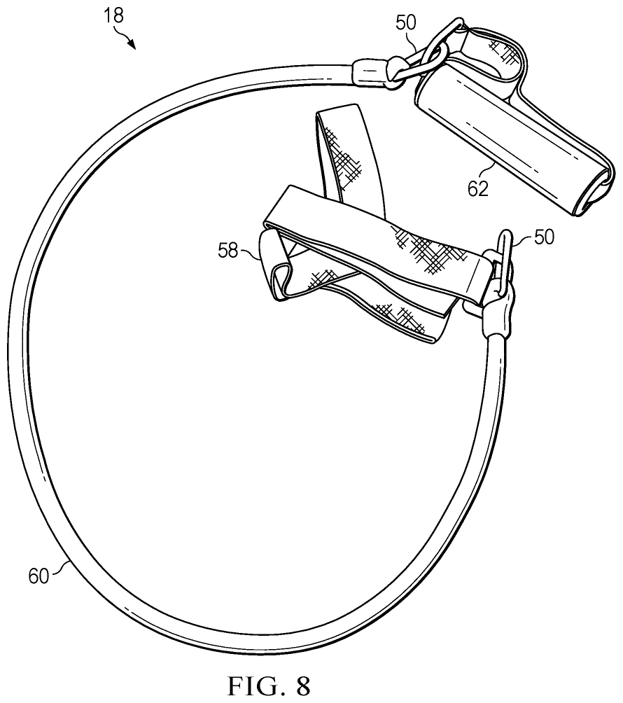

FIG. 8 is an isolated view of an optional, supplemental arm resistance element 18. In the depicted embodiment, the length of elastomeric material 60 (e.g., tubing), may be different than used in either the arm resistance element 14 or the leg resistance element 16. Referring to FIGS. 6-8, a closed loop 54 attached to the foot strap 58 may permit the foot strap 58 to be removably attached to one of the end clips 50 of elastomeric material 60. The end clips 50 of the elastomeric material 60 of the supplemental arm resistance element 18 would be very likely to be identical to said end clips 50 of the elastomeric material 56 of the foot strap 58. The length and diameter of the elastic material 60 for the supplemental arm resistance element 18 may be varied and selected by the user, so that a selected amount of resistance may be provided against a length-wise stretching force. In fact, the supplemental arm resistance element 18 could be constructed from one of the lengths of elastomeric material 48, 56. A hand strap 52 or a hand grip element 62 may be affixed to one of the end clips 50 of the elastomeric material 60, and a foot strap 58 may be affixed to the other end (as shown in FIG. 8). Hand and/or foot straps may also individually or collectively be referred to herein as “appendage strap(s).”

Any embodiment of the present invention may include any of the features of the other embodiments of the present invention. The exemplary embodiments disclosed herein are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. An apparatus, wearable by a user, comprising:
a harness device, configured to be to worn on an upper torso, the harness device having a plurality of harness straps that are adjustable to provide targeted pressure to the user, the device capable of achieving posture correction of the user by aligning the user's back and shoulders.

2. The apparatus of claim 1, further comprising:
a plurality of resistance elements, each of which comprises a length of elastomeric material with a fastening means affixed at each end thereof;
a plurality of appendage straps, configured for receiving a hand or foot of the user, each appendage strap having a fastening means complementary to the fastening means on the resistance elements;
a plurality of fastening means, complementary to the fastening means on the resistance elements, arranged on the harness device;
wherein the user is permitted to exercise one or more appendages by anchoring one end of one of the resistance elements to the harness device and inserting a hand or foot into a strap attached at an opposite end of the resistance element.

3. The apparatus of claim 1, wherein a length of each harness strap of the plurality of harness straps is adjustable to accommodate different torso sizes.

4. The apparatus of claim 1, wherein a length of each appendage strap of the plurality of appendage straps is adjustable to accommodate different hand or foot sizes.

5. An apparatus, wearable by a user, comprising:
a harness device, configured to be to worn on an upper torso of the user, the harness device having a plurality of adjustable harness straps;
at least one resistance element, configured to be connected to both the harness device and an appendage of the user; and
wherein the resistance element is capable of providing targeted pressure to the appendage of the user.

6. The apparatus of claim 5, further comprising:
an appendage strap, configured to permit attachment of the at least one resistance element to the appendage of the user;
wherein a length of each harness strap of the plurality of harness straps is adjustable to accommodate different torso sizes; and
wherein a length of the appendage strap is adjustable to accommodate different hand or foot sizes.

7. The apparatus of claim 6, wherein a belt loop permits adjustment of the length of at least one harness strap of the plurality of harness straps.

8. The apparatus of claim 5, wherein the plurality of adjustable harness straps include a horizontal strap and a plurality of vertical straps.

9. The apparatus of claim 5, wherein the at least one resistance element includes a pair of arm resistance elements and a pair of leg resistance elements.

10. The apparatus of claim 9, wherein an appendage strap is connected to each resistance element of the pair of arm resistance elements and the pair of leg resistance elements.

11. The apparatus of claim 9, wherein the at least one resistance element further includes a supplemental resistance element.

12. The apparatus of claim 5, wherein the at least one resistance element is connected to the harness device at a closed loop of the harness device.

13. The apparatus of claim 8, wherein the plurality of vertical straps includes a shoulder strap.

14. The apparatus of claim 8, wherein at least one strap selected from the group of straps comprising the horizontal strap and the plurality of vertical straps includes a hook-and-loop fastener.

15. The apparatus of claim 5, wherein the harness device further comprises an x-shaped strap arrangement configured to be positioned proximate to the user's back.

16. The apparatus of claim 2, wherein the elastomeric material is formed as tubing, and end clips are affixed to each end of the tubing.

17. An apparatus, wearable by a user, comprising:
a harness device, configured to be to worn on an upper torso of the user, the harness device comprising:
a plurality of harness straps, including a horizontal strap, a plurality of vertical straps, and an x-shaped strap arrangement configured to be positioned proximate to the user's back;
a plurality of closed loops;
a plurality of resistance elements, each comprising a length of elastomeric material having a first clip affixed to a first end thereof, and a second clip affixed to a second end thereof;
wherein each resistance element is capable of providing targeted pressure to an appendage of the user;
wherein the first clip of each resistance element permits the first end to be affixed to one of the plurality of closed loops, and the second clip of each resistance element permits the second end to be affixed to an appendage strap; and
wherein a length of each harness strap of the plurality of harness straps is adjustable to accommodate different torso sizes.

18. The apparatus of claim 17, wherein the plurality of resistance elements includes a pair of arm resistance elements and a pair of leg resistance elements.
